(19) United States
(12) Patent Application Publication
(10) Pub. No.: US 2011/0028709 A1
(43) Pub. Date: Feb. 3, 2011

(54) METHODS FOR STERILIZING GLUCANS

(76) Inventors: Mark Gerald Deacon-Shaw, Erina (AU); Reinhard Koenig, Rockville, MD (US)

Correspondence Address: EDWARDS ANGELL PALMER & DODGE LLP, P.O. BOX 55874, BOSTON, MA, 02205, US

(21) Appl. No.: 12/734,443

(22) PCT Filed: Oct. 29, 2008
(86) PCT No.: PCT/AU2008/001598

§ 371 Date: Oct. 25, 2010

Related U.S. Application Data
(60) Provisional application No. 60/984,045, filed on Oct. 31, 2007.

(51) Int. Cl. C08B 37/00; A61L 2/04

(52) U.S. Cl. 536/123.12; 422/1

(57) ABSTRACT

The present invention provides a method for sterilizing glucan, the method comprising subjecting the glucan to dry heat sterilization.

### TECHNICAL FIELD

The present invention relates to methods for sterilizing glucans that are intended for inclusion in compositions for administration to humans and/or animals. The method generally involves the use of dry heat.

### BACKGROUND OF THE INVENTION

Glucans are polysaccharide molecules consisting of glucose subunits. Beta-glucan is a potent stimulator of the immune system because of its ability to enhance the effect of macrophages. Beta-glucan may also moderate glycaemic response, assist in wound healing, lower serum cholesterol levels and possess anti-tumour activity. In light of the utility of beta-glucan in particular, therapeutic compositions comprising glucans have been commercially available for a number of years.

Beta-glucans occur in a variety of organisms including microorganisms, basidiomycetes and plants, and are typically located in the cell walls of such organisms. Beta-glucan is therefore usually isolated from the environment in which it naturally occurs, and subsequently included in therapeutic or health supplemental compositions.

An example of the isolation of glucan from a natural source can be found in U.S. Pat. No. 6,242,594, which discloses a process for isolating a microparticulate beta-glucan from a naturally occurring glucan source by a series of extraction steps. The particular glucan obtained from this process (poly-(1,3)-beta-D-glucopyranosyl-(1,6)-beta-D-glucopyranose) has been found to be therapeutically effective when administered, for example, to subjects suffering from a bone fracture, ulcers caused by physical trauma, impaired blood flow, infections or neoplasia, or in persons in need of enhancement of fixation of implanted orthopaedic devices to bone. It is thought that poly-(1,3)-beta-D-glucopyranosyl-(1,6)-beta-D-glucopyranose may modulate the natural cascade of wound healing activities of several cell populations when applied directly to a wound surface. Such cell populations include macrophages, fibroblasts, vascular endothelial cells, epithelial cells and neutrophils.

Because compositions comprising glucans are administered to humans and animals, it is vital that the constituent glucan is free from potentially harmful contaminants and byproducts (e.g. bacterial spores) that may have survived the extraction process. It is therefore desirable to sterilize beta-glucan following any extraction process prior to its inclusion in a therapeutic or health supplemental composition. Indeed most therapeutic compositions for in vivo administration to humans are sterilized, and in many cases sterilization is required to meet stringent regulatory authority (e.g. FDA) requirements.

A well known and effective method of sterilization involves subjecting active pharmaceutical components to steam sterilization, which generally involves heating in an autoclave employing saturated steam at a pressure of approximately 15 psi so as to achieve a chamber temperature of about 121° C. However, in the case of glucans this method has proved to be ineffective and unacceptable due to the swelling and agglomeration of the glucan particles. Further drying and milling of the glucan product would be required in this instance which adds to production time and costs, which is obviously undesirable. In addition, this particular method may also lead to a reduction in the pharmacological activity of the glucan.

Against this background, there is a need for a method of sterilizing glucans for inclusion in products intended for administration to humans or animals which addresses is one or more of the above-mentioned disadvantages.

### SUMMARY OF THE INVENTION

In a first aspect, the present invention provides a method for sterilizing glucan, the method comprising subjecting the glucan to dry heat sterilization.

In a second aspect, the present invention provides a method for the preparation of a sterile glucan, the method comprising subjecting the glucan to dry heat sterilization.

In a third aspect, the present invention provides a glucan whenever obtained by the process of the first or second aspects.

The glucan may be any glucan that is intended for inclusion in a composition for administration to humans or animals, for example therapeutic or health supplemental compositions.

The glucan may be soluble, insoluble, particulate or microparticulate glucan.

The glucan may be beta-glucan.

The glucan may be beta-(1,3)(1,6) glucan.

The glucan may be a particulate branched beta-(1,3)(1,6) glucan that is essentially free of unbranched beta-(1,3) glucan.

The glucan may be microparticulate poly-(1,3)-beta-D-glucopyranosyl-(1,6)-beta-D-glucopyranose.

The glucan may be a glucan obtained from the process described in U.S. Pat. No. 6,242,594.

The glucan may be obtained from a cellular glucan source.

The cellular glucan source may be a microorganism.

The microorganism may be Saccharomyces cerevisiae.

The dry heat sterilization may involve subjecting the glucan to a temperature of between about 140° C. and about 180° C.

The dry heat sterilization may be performed for a period of between about 20 minutes and about 12 hours, or between about 1 hour and about 10 hours. In one embodiment, the dry heat sterilization may be performed at a temperature of about 160° C. for a period of about 2 to 4 hours.

The method may be performed at atmospheric pressure.

The method may be performed under positive pressure.

The method may be performed in an oven.

The oven may be an oven that is able to circulate air, for example a convection oven such as a fan forced oven.

The method may further comprise the step of allowing the glucan to cool following dry heat sterilization in a vessel equipped with a HEPA filter.

In a fourth aspect, the present invention provides a method for isolating a glucan from a glucan-containing cellular source, the method including the step of sterilizing the glucan by dry heat sterilization.

The glucan may be a glucan as defined above in relation to the first, second or third aspects.

The glucan-containing cellular source may be Saccharomyces cerevisiae.

The method for isolating a glucan may be a method as described in U.S. Pat. No. 6,242,594.

### BRIEF DESCRIPTION OF THE FIGURES

FIG. 1 shows TNF-α release from macrophage cultures: (i) stimulated with two microparticulate beta-glucan samples following dry heat sterilization (IGP006.004R and IGP06.005R); (ii) in the presence of bakers yeast, and (iii) with no stimulation (control).

### Definitions

In the context of this specification, the terms “a” and “an” are used herein to refer to one or to more than one (i.e. to at least one) of the grammatical object of the article. By way of example, “an element” means one element or more than one element.

In the context of this specification, the term “comprising” means “including principally, but not necessarily solely”. Furthermore, variations of the word “comprising”, such as “comprise” and “comprises” have correspondingly varied meanings.

In the context of this specification, the term “dry heat sterilization” is understood to mean a method whereby a sample to be sterilized is heated in an environment wherein the humidity level is less than 100%, for a period of time necessary to render the sample sterile.

In the context of this specification, the term “glucan” is understood to mean a polysaccharide of glucose monomers linked by glycosidic bonds.

In the context of this specification, the term “sterile” is understood to mean that the glucan product meets the standards required by the Sterility test as per the US Pharmacopeia.

In the context of this specification, the term “microparticulate” is understood to mean in the form of particles not more than 40 μm in size.

### DETAILED DESCRIPTION OF THE INVENTION

The present invention is based on the surprising discovery by the inventor that glucan can be rendered sterile by dry heat sterilization, and that such a method does not adversely impact the pharmacological properties of the glucan. These observations were surprising and unexpected in light of the fact that the wet steam sterilization method was unsuitable, and also because of the significantly higher temperatures and times required when using dry heat sterilization.

The dry heat sterilization may involve subjecting the glucan to a temperature of about 160° C., however temperatures in the range of about 140° C. to 180° C. may also be used. Accordingly, the dry heat sterilization may be performed at a temperature between about 140° C. and about 180° C., or between about 142° C. and about 180° C., or between about 144° C. and about 180° C., or between about 146° C. and about 180° C., or between about 148° C. and about 180° C., or between about 150° C. and about 180° C., or between about 152° C. and about 180° C., or between about 154° C. and about 180° C., or between about 156° C. and about 180° C., or between about 158° C. and 180° C., or between about 160° C. and about 180° C., or between about 162° C. and about 180° C., or between about 164° C. and about 180° C., or between about 166° C. and about 180° C., or between about 168° C. and about 180° C., or between about 170° C. and about 180° C., or between about 172° C. and about 180° C., or between about 174° C. and about 180° C., or between about 176° C. and about 180° C., or between about 140° C. and about 178° C., or between about 140° C. and about 176° C., or between about 140° C. and about 174° C., or between about 140° C. and about 172° C., or between about 140° C. and about 170° C., or between about 140° C. and about 168° C., or between about 140° C. and about 166° C., or between about 140° C. and about 164° C., or between about 140° C. and about 162° C., or between about 140° C. and about 160° C., or between about 140° C. and about 158° C., or between about 140° C. and about 156° C., or between about 140° C. and about 154° C., or between about 140° C. and about 152° C., or between about 140° C. and about 150° C., or between about 140° C. and about 148° C., or between about 140° C. and about 146° C., or between 142° C. and about 178° C., or between about 144° C. and about 176° C., or between about 146° C. and about 174° C., or between about 148° C. and 172° C., or between about 150° C. and about 170° C., or between about 152° C. and about 168° C., or between about 154° C. and about 166° C., or between about 156° C. and about 164° C., or between about 158° C. and 162° C., or between about 155° C. and about 165° C., or between about 157° C. and about 162° C.

The dry heat sterilization may be performed for a period of between about 20 minutes and about 5 hours, or between about 45 minutes and about 4 hours, or between about 1 hour and about 4 hours, or between about 1.25 hours and about 4 hours, or between about 1.5 hours and about 4 hours, or between about 1.75 hours and about 4 hours, or between about 2 hours and about 4 hours, or between about 2.25 hours and about 4 hours, or between about 2.5 hours and about 4 hours, or between about 2.75 hours and about 4 hours, or between about 3 hours and about 4 hours, or between about 3.25 hours and about 4 hours, or between about 3.5 hours and about 4 hours, or between about 30 minutes and about 4 hours, or between about 1 hour and about 3.5 hours, or between about 1 hour and about 3.25 hours, or between about 1 hour and about 3 hours, or between about 1 hour and about 2.75 hours, or between about 1 hour and about 2.5 hours, or between about 1 hour and about 2.25 hours, or between about 1 hour and about 2 hours, or between about 45 minutes and about 3 hours, or between about 1 hour and about 3 hours, or between about 1 hour and about 2.5 hours or between about 30 minutes and about 3 hours, or between about 30 minutes and about 2.5 hours or between about 1.5 hours to about 3 hours, or about 1.5 hours to about 2.75 hours, or about 1.5 hours to about 2.5 hours. The dry heat sterilization may be performed for a period of less than 20 hours, or less than 15 hours, or less than 12 hours, or less than 10 hours, or less than 9 hours, or less than 8 hours, or less than 7 hours, or less than 6 hours, or less than 5 hours, or less than 4 hours. The dry heat sterilization may be performed for a period of between about 1 hour and about 15 hours, or between about 1 hour and about 10 hours, or between about 1 hour and about 8 hours.

The dry heat sterilization may be carried out for an amount of time sufficient to produce a sterile glucan that maintains the desired pharmacological properties. Generally, as the temperature to which the glucan is subjected is increased, the lower the amount of time required to achieve sterilization.

In one embodiment of the invention, the method may comprise subjecting the glucan to a temperature of between about 140° C. and 180° C. for less than about 20, 15, 12, 10, 9, 8, 7, 6, 5, 4, or 3 hours.

In another embodiment of the invention, the method may comprise subjecting the glucan to a temperature of between about 140° C. and 180° C. for a period of between about 1 hour and about 10 hours.

In a further embodiment of the invention, the method may comprise subjecting the glucan to a temperature of between about 140° C. and about 170° C. for a period of between about 1 to 5 hours, such that where 170° C. is used, the time period is about 1 to 2.5 hours, where 160° C. is used, the time period is about 2 to 3.5 hours, where 150° C. is used, the period is about 2.5 to 4 hours, and where 140° C. is used, the time period is about 3 to 4 hours.

In an embodiment of the invention, the method may comprise subjecting the glucan to a temperature of between about 145° C. and about 170° C. for a period between about 1 hour and about 4.5 hours.

In an alternative embodiment, the method may comprise subjecting the glucan to a temperature of between about 155° C. and 165° C. for a period of between about 1 hour and about 3.5 hours, or between about 1.25 hours and about 3 hours, or between about 1.5 hours and about 2.75 hours.

The method of the invention may be carried out in such a manner that all parts of the glucan sample that is to be sterilized reaches, and is maintained within, the desired temperature range for the desired time. The present process may be carried out batch wise.

The method of the invention is suitable for the sterilization of any type of glucan, for example soluble glucans, insoluble glucans or particulate glucans, including microparticulate glucans. In one embodiment of the invention the glucan is a beta-glucan, for example beta-(1,3)(1,6) glucan, particulate branched beta-(1,3)(1,6) glucan that is essentially free of unbranched beta-(1,3) glucan or microparticulate poly-(1,3)-beta-D-glucopyranosyl-(1,6)-beta-D-glucopyranose. In an alternative embodiment, the glucan may be GLYC-101 or glucoprime™ (a high molecular weight microparticulate glucan with a high degree of side-branching), both of which are available from Glycotex, Inc, Rockville, Md. In a further embodiment of the invention, the glucan may be a glucan that is obtained from the isolation process described in U.S. Pat. No. 6,242,594.

### Modes for Performing the Invention

The method of the present invention may conveniently be performed in any vessel that is capable of maintaining a constant temperature, in this context, “constant” means maintaining a temperature of x ° C.±3° C. The vessel may be under atmospheric conditions, or alternatively may be under an atmosphere of inert gas, such as nitrogen or argon. The vessel may be maintained under a slight positive pressure during sterilization, however the method may be performed in an equally effective manner under atmospheric pressure conditions. Further, it is not necessary to exert any control over humidity in the vessel. An example of a suitable vessel in which to carry out the method of the invention is the Pyromega range of dry heat ovens available from Celester Technologies, Barcelona, Spain, although it is to be noted that the method may also be carried out in a more basic laboratory-type oven.

When performing the method of the invention, the glucan to be sterilized may be inserted into the vessel following pre-heating of the vessel to the desired temperature. Alternatively, the glucan to be sterilized may be placed into the vessel at room temperature, prior to heat being introduced. In the latter case, the exact time over which the glucan is exposed to the desired temperature will depend on the heating rate of the vessel, however this can be monitored if desired by observing when the vessel reaches the desired temperature once the heat has been activated. As such, in some embodiments where a time period of exposure to a given temperature is stated, the actual time period over which the glucan is exposed to this temperature may in fact be between approximately 10 minutes and 45 minutes longer (depending on how rapidly the vessel reaches the desired temperature), in the situation where the glucan sample is placed in the vessel prior to the commencement of heating.

In one embodiment, the method may be performed in an oven as follows. The sample of glucan to be sterilized is placed on a tray (for example a pyrex drying tray), and the tray inserted into the oven which is at room temperature. The glucan may be placed evenly on the tray at a depth of about 20 mm. It is preferred that the tray be placed in a position near the centre of the oven so as to optimise the flow of hot air around the tray.

The oven, which is equipped with a thermometer or a thermocouple, is set to a temperature of between about 140° C. and 170° C. The oven may be equipped with a mechanical air circulation device (for example a fan) so as to ensure equal air temperature distribution throughout the oven. In addition, hot air flowing over surfaces reduces microorganism resistance by way of dehydration, which may result in the time required to achieve sterilization being reduced.

The tray is left for a period of about 1 to 3 hours (about 1 hour if the oven temperature is 170° C., about 2 hours if the oven temperature is 160° C., about 2.5 hours if the oven temperature is about 150° C. and about 3 hours if the oven temperature is about 140° C.). Once the temperature in the oven reaches the desired temperature, it is monitored throughout the time period over which the method is performed so as to ensure that the temperature remains constant. Once the sterilization is complete, the tray is removed from the oven and allowed to cool under atmospheric conditions. Alternatively, the tray may be removed from the oven and immediately placed under sterile conditions and allowed to cool at room temperature. In one embodiment, the tray may be placed in a container or vessel equipped with a HEPA filter, and allowed to cool at room temperature. In another embodiment, cooling may be achieved by introducing ambient air into the oven via a HEPA filter and allowing this air to circulate to facilitate cooling. Once the cooling is complete, the sterilized glucan may be transferred to an appropriate sterile jar for storage.

It is desirable to clean and maintain the vessel in which the method is performed according to the manufacturer's recommendations. Cleaning and preventative maintenance should reduce possible equipment malfunctions that could lead to the sterilization method being compromised. There is however minimal preventative maintenance required for hot air ovens, and in this regard monitoring the accuracy of the thermostats, the fan motor (if fitted) and the electrical cords and plugs is really all that is necessary.

Sterilization indicators may be used in accordance with the method of the invention so as to ensure that the required exposure conditions of the glucan sample have been met at the point of placement of the indicator. An example of such an indicator is the 3M Comply™ dry heat chemical indicator available from 3M Company St. Paul, Minn.

Biological indicators may also be used to confirm the performance of the sterilization method. These types of indicator devices are based on microbial spores (typically pure strains of the heat-resistant microbe Geobacillus stearothermophilus), and contain a liquid growth medium and a growth indicator. Following completion of the sterilization method, an internal glass ampule is shattered releasing the spores into the growth medium. The vial is then incubated at about 50° C. for 48 hours. If the spores were not destroyed as a result of the sterilization method, the medium will undergo a colour change during the incubation.

The dry heat sterilization method of the invention may be used in conjunction with any process for preparing or isolating glucan, typically as the final step of such a process and/or prior to the inclusion of glucan in a product for administration to humans or animals. For example, the dry heat sterilization method may form part of the process for isolating glucan that is described in U.S. Pat. No. 6,242,594, the disclosure of which is incorporated herein by reference. In this particular example, the dry heat sterilization method may be carried out as part of the process set out in Example 1 of U.S. Pat. No. 6,242,594 following the spray drying of the isolated glucan product.

By including the dry heat sterilization step in a process for isolating glucan for example, one is able to conveniently prepare a sterile glucan from a naturally occurring glucan source that is able to be included in a therapeutic or health supplemental composition.

### EXAMPLES

### Example 1

### Dry Heat Sterilization Versus Wet Steam Sterilization

Three 100 gram batches of a microparticluate beta-glucan were subjected to the following sterilization conditions:

1) Wet steam sterilization at 121° C. for 20 minutes, where the glucan was present in an autoclave bag;

2) Wet steam sterilization at 135° C. for 5 minutes, where the glucan was present in an autoclave bag; and

3) Dry steam sterilization comprising subjecting the glucan to 160° C. for 1 hour on a drying tray.

Immediately following each experiment, the samples were transferred to a HEPA booth and allowed to cool to room temperature. Whilst the samples subjected to conditions 1) and 2) were found to contain less than 10 CFU per gram, the samples contained significant amounts of agglomerates and were unacceptable for use in therapeutic and health supplemental compositions.

The sample subjected to condition 3) also contained less than 10 CFU per gram, however no agglomeration occurred. It appeared that the dry heat sterilization had no m observable physical effect on the glucan.

### Example 2

### TNF-α Release Following Heat Sterilization

In order to assess the pharmacological activity of microparticluate beta-glucan samples following dry heat sterilisation, the release of TNF-α from macrophage cultures following stimulation with two batches of sterilized glucan was measured using ELISA. Experimental details follow.

### 2.1 Cell Culture and Culture Medium

Two types of medium were prepared. The “Complete MØ Medium” was prepared with 450 mL of RPMI 1640 supplemented with 50.0 mL of Human Sera Type AB, 5.0 mL of 200 mM L-Glutamine and 5.0 mL of Penicillin Streptomycin. The “MØ Medium-10% FBS” was formulated by adding 50 mL of Fetal Bovine Serum (FBS) and 5.0 mL of 200 mM L-Glutamine to 450 mL of RPMI 1640.

Monocytes isolated from human donors were seeded in as many 24 well plates as possible at 5.0×10⁵ cells per well. The culture plates were incubated at 37° C. with 5% CO₂ for seven days. The cultures were fed on day three or four. On day 7, the non-adherent cells from the cultures were removed. The culture medium in each well was mixed thoroughly with a 1 mL pipette and removed from the cultures. The adherent monocytes were washed with 1 mL of PBS, again mixing the wash with a 1 mL pipette to remove any remaining non-adherent cells. The cultures are then stimulated with the dry sterilized glucan.

### 2.2 Glucan Suspensions

Suspensions of the dry sterilized glucan were prepared as stock solutions by using a balance to weigh out 1-2 mg of the glucan and then adding the appropriate amount of PBS to reach a 1 mg/mL concentration. Glucan suspensions and corresponding placebos were also prepared from gel formulations. The gels (glucan and placebo) were prepared using a balance to weigh out the gel material and then adding the appropriate volume of MØ Medium-10% FBS to reach a 1 mg/mL concentration of gel per mL of MØ Medium-10% FBS, which is equivalent to a 0.01 mg/mL glucan concentration per mL of MØ Medium-10% FBS. The spent medium in the culture well was removed and replaced with 1 mL of the 1 mg/mL gel suspension in MØ Medium-10% FBS for a final concentration of 0.01 mg/mL per well.

### 2.3 Commercial Glucan Control Suspensions

Glucan consisting of Baker's Yeast (Sigma G5011) suspensions were prepared as stock solutions by using a balance to weigh out 1-2 mg of the material and then adding the appropriate amount of PBS to reach a 1 mg/mL concentration.

### 2.4 Addition to the Macrophage Cultures

After removal of the non-adherent cells in the 24 well plate cultures, 0.90 mL of MØ Medium-10% FBS or Complete MO Medium was added to all the wells. The appropriate amount of the 1 mg/mL glucan suspension (Lots 1GP06.004R and 1GP06.005R) were added to the corresponding wells for a final concentration of 100 μg/mL Positive controls (for stimulation) consisting of 100 μg/mL of Baker's Yeast (Sigma G5011) were also included on each plate. For the negative control (“Control No Stim.”), 0.1 mL PBS was added. The cultures were observed microscopically. Photographs were taken of the cells at 24 hours post stimulation and immunofluorescence staining was conducted to measure the cell surface marker CD14. Supernatants were harvested from each well at 4 hours or 24 hours post stimulation and immediately frozen at −30° C. for later analysis by ELISA.

### 2.5 TNF-α ELISA

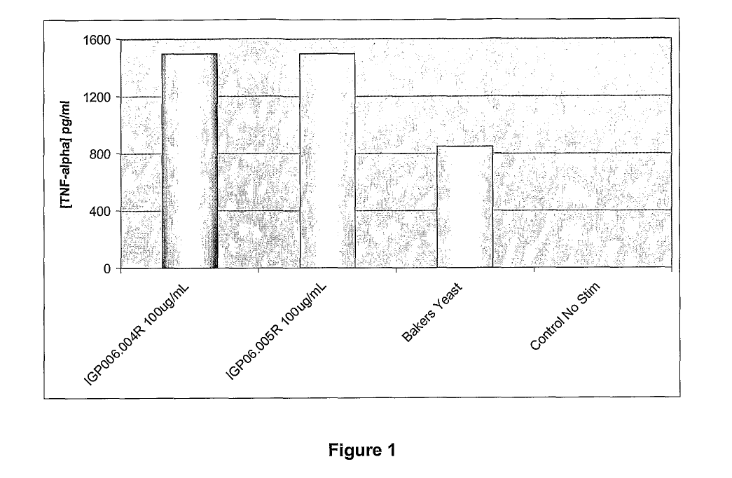

TNF-α levels were measured using ELISA kits obtained from R&D Systems, Inc. Frozen MØ culture supernatants were thawed at room temperature for approximately 1-1.5 hours and added to the ELISA plate. All the ELISAs were performed according to the manufacturer's instructions. Samples were assayed undiluted or they were diluted between 1:10 and 1:10,000. The results are depicted in FIG. 1.

As can be seen in FIG. 1, stimulation with both dry sterilized glucan products lead to TNF-α release in significant amounts as compared to stimulation with bakers yeast. This result demonstrates that glucan sterilized in accordance with the method of the present invention retains its desired pharmacological activity.

From a reading of the description above in light of the appended drawings, it will be obvious to those with ordinary skill in the art that further modifications and changes may be made to the embodiments described herein without departing from the spirit and scope of the present invention as claimed.

1. A method for sterilizing a glucan or preparation of a sterile glucan, the method comprising subjecting the glucan to dry heat sterilization.

2. (canceled)

3. The method of claim 1, wherein the glucan is any glucan that is intended for inclusion in a composition for administration to humans or animals.

4. The method of claim 1, wherein the glucan is soluble, insoluble, particulate or microparticulate glucan.

5. The method of claim 1, wherein the glucan is beta-(1,3)(1,6) glucan.

6. The method of claim 1, wherein the glucan is a particulate branched beta-(1,3)(1,6) glucan that is essentially free of unbranched beta-(1,3) glucan.

7. The method of claim 1, wherein the glucan is microparticulate poly-(1,3)-beta-D-glucopyranosyl-(1,6)-beta-D-glucopyranose.

8. The method of claim 1, wherein the dry heat sterilization is performed for an amount of time sufficient to produce a sterile glucan.

9. The method of claim 1, wherein the dry heat sterilization involves subjecting the glucan to a temperature of between about 140° C. and about 180° C.

10. The method of claim 1, wherein the dry heat sterilization is performed for a period of between about 20 minutes and about 8 hours.

11. The method of claim 1, wherein the dry heat sterilization is performed at a temperature of between about 155° C. and about 165° C. for a period of between about 2 to 4 hours.

12. The method of claim 1, wherein the dry heat sterilisation is performed at a temperature of about 160° C. for a period of about 2 to 3 hours.

13. The method of claim 1, wherein the method further comprises the step of allowing the glucan to cool following dry heat sterilization in a vessel equipped with a HEPA filter.

14. A glucan, whenever obtained by the process of claim 1.

15. A method for isolating a glucan from a glucan-containing cellular source, the method including the step of sterilizing the glucan by dry heat sterilization.

16. (canceled)
